(19) United States
(12) Patent Application Publication
(10) Pub. No.: US 2003/0057590 A1
(43) Pub. Date: Mar. 27, 2003

(54) PROCESS FOR MANUFACTURING COMPONENTS MADE OF FIBER-REINFORCED THERMO- PLASTIC MATERIALS AND COMPONENTS MANUFACTURED BY THIS PROCESS

(76) Inventors: URS LOHER, WIL (SE); JORG MAYER, HORNORECNTIXON (CH); ROGER TOGININI, KLETTYAU (DE); THOMAS WEGENER, SPEICHER (CH); ERIC WINERMANTEL, FISILISBACH (CH)

Correspondence Address: HELFGOTT & KARAS, EMPIRE STATE BUILDING, SUITE 6024, NEW YORK, NY, 10118

(21) Appl. No.: 08/849,746

(22) PCT Filed: Dec. 18, 1995
(86) PCT No.: PCT/EP95/04992

(30) Foreign Application Priority Data
Dec. 19, 1994 (DE) P 44 45 305.1
Dec. 19, 1994 (DE) P 44 45 307.8

(51) Int. Cl. B29C 70/42; B29C 43/02

(52) U.S. Cl. 264/157; 264/134; 264/136; 264/137; 264/322; 264/324

(57) ABSTRACT

An extended component made of fibre-reinforced thermoplastic materials, in particular a screw (1) that contains a corresponding proportion of fibres. Carbon fibres shaped as endles fibres extend in an at least approximately parallel direction to the centre line of the screw (1) in the area of the head (2) of the adjacent thread turns of the shaft (5). At the surface of the remaining part of the threaded portion, the fibre follow the contour of the thread in the axial direction of the part. The fibres in the core of this section next to the end of the screw are distrubuted in an increasingly random manner towards the free end of the screw.

The invention relates to a process for manufacturing components made of fiber-reinforced thermoplastic materials, where a blank formed of a short, long, and/or endless fiber and a thermoplastic material is first pre-finished, and this blank is brought into the final form of the component in a negative mold, under pressure, in a hot-forming process, a process for manufacturing components which are under tensile, bending, and/or torsion stress, made of fiber-reinforced thermoplastic materials, where a blank formed with a fiber proportion of more than 50 volume-% and with at least predominant use of endless fibers and a thermoplastic material is first pre-finished, and this blank is brought into the final form of the component in a negative mold, under pressure, in a hot-forming process, as well as components manufactured by one of these processes.

Components made of fiber-reinforced thermoplastic materials are mostly used as connecting elements. These components are intended to replace metal screws, for example. Particularly for use in medical technology, in other words as bone screws, for example, screws made of fiber-reinforced thermoplastic materials are significantly better suited, since they are compatible in structure with the bone, no problems with corrosion resistance occur, the weight can be reduced as compared with metal screws, and the usual medical examination methods are not hindered, in contrast to the use of metal.

Screws and threaded rods made of fiber-reinforced thermoplastic materials have already become known, where the screw blanks are produced either by coextrusion or by means of a multi-component injection molding process. In this known embodiment (DE-A-40 16 427), circular solid rods produced by means of coextrusion are used as the starting material. For the core region, thermoplastic granulate with long fibers, length 5-10 mm, is prepared in an extruder; for the other region, thermoplastic material with short fibers is prepared in a second extruder. This results in a starting material in which a coaxial arrangement with inner long fibers and outer short fibers is present. The long fibers in the inner core region are predominantly directed in an axial direction, by means of an extrusion flow process, while the short fibers in the outer region transfer shear forces into the thread turns. The thread turns are produced by subsequent cold-forming, e.g by means of thread roll heads or machines. Such cold-forming is made possible by the use of short fibers, but reduced strength values result, in particular, from the arrangement of such short fibers in the thread region.

In a process in accordance with DE-T2-68919466, a blank is inserted into a two-part mold and formed in it. The cold blank is laid into a mold cavity, heated, expanded, and cooled. Therefore, forming is possible only to a limited degree, and in addition, it is almost impossible to influence the alignment of the fibers, or at least not possible to do so in a pre-determined manner.

The present invention has now set itself the task of creating a process for manufacturing components made of fiber-reinforced thermoplastic materials, with which an optimum adaptation to the components' purpose of use is possible. Furthermore, it is the task of the invention to create components manufactured according to this process, with which the force introduction and distribution, i.e. the rigidity can be adapted to the composition of the body interacting with the component, in particular manner.

The process according to the invention therefore provides that the blank is first heated to forming temperature in a heating stage, and then pressed into the negative mold by means of extrusion. The fibers, which are distributed over the entire cross-section of the blank, are oriented and distributed in a manner that can be controlled, in very targeted manner, by means of the subsequent extrusion process. The fiber orientation and fiber distribution and therefore the mechanical properties of a component manufactured according to this process can therefore be specifically characterized and brought into relation with the process parameters of the manufacturing process. By means of extrusin, the fiber orientation can be additionally controlled, so that different strength values can also be achieved over the length of a corresponding component.

In a process using more than 60 volume-% endless fibers, the blank is also heated to forming temperature in a heating stage, and then pressed into the negative mold by means of extrusion. Specifically when using a great density of endless fibers, the rigidity and the strength of a component to be manufactured can be controlled in very targeted manner. Particularly in the case of components with a complex shape, the precise predictability of the optimum fiber progression and the optimum fiber density in a certain region has an advantageous effect.

Furthermore, it is proposed that the blank is pre-finished as rod materials, and cut to the length required for the final components before the hot-forming process. The materials pieces required for the final components are cut off the pre-finished rod material, and subsequently brought into the hot-forming process. In other words, the method of procedure is similar to that of extrusion of metal parts.

Specifically when using endless fibers, it is provided that fibers with a length that correspondes at least to the length of the blank for the final component are used. This makes it possible to achieve even more improved rigidity and strength.

It would also be possible for a blank composed of layers with different fiber orientation in its lengthwise direction to be formed. In other words, it is possible to cover uncounted new areas of use with the process according to the invention, since the work is always aimed at a very specific purpose of use for the components to be manufactured, and a precisely pre-determined strength and rigidity can be achieved.

In this connection, it is also possible that a blank is formed from more than one polymer laminate, e.g. with several layers with a different matrix material and a different arrangement and/or different volume-% proportion and/or different fiber material and/or different lengths of the fibers. It is also possible to achieve a precise adaptation to the final requirements for the component to be manufactured by means of such measures.

In this connection, it can also be advantageous if the blank is formed into the final component by means of a push-pull extrusion process. The blank, cut off from the rod material, is formed in a corresponding extrusion mold, where the so-called push-through process according to DIN 8583 can be used. In the push-pull extrusion process, the blank is formed into the final component, in the negative mold, in several back-and-forth movements. Particularly in manufacturing strip-shaped or plate-shaped components, this process has a particularly positive effect.

As compared with extrusion or push-pull extrusion of metal parts, a significant characteristic of differentiation that is provided is that in the extrusion or push-pull extrusion process, the blank is heated to a forming temperature of 350-450° C., for example, in a heating stage, and then pressed into the negative mold, where cooling below the glass transition temperature of the thermoplastic material, e.g. 143° C., takes place during a post-pressure phase. For processing the fiber-reinforced thermoplastic materials, the extrusion process known for metal parts is changed in that the blank is formed not at room temperature, but rather above the melting or plastification temperature of the matrix material.

Furthermore, it is advantageous that during the hot-forming process, carbon or graphite is used as a release agent. Such a release agent was obviously not used in forming thermoplastic materials until now. Here there is the additional particular advantage that graphite, for example, is biocompatible, in contrast to the coatings or release agents otherwise usually used for plastics, so that components for the medical sector are particularly suitable for this. Furthermore, it is provided, in accordance with the process according to the invention, that a blank made of PAEK (polyaryl ether ketones) reinforced with carbon fibers is processed. It has been shown that by means of the use specifically of such a material, the tensile strength of the component manufactured in this manner is approximately 30%, on the average, below the tensile strength of comparable steel components. For the area of use of such components made of fiber-reinforced thermoplastic materials, however, this is a more than sufficient strength, since it must always be taken into consideration what materials such a component is intended to interact with. Particularly when used in medical technology, in other words as bone screws, for example, or as plate-shaped or rail-shaped components, a correspondingly high fracture resistance is certainly sufficient, since such components already possess almost three times the available fracture resistance of a bone.

In the process according to the invention, it is furthermore provided that at least part of the fibers run parallel to the axis of the blank. It is also possible, however, that at least a portion of the fibers have an orientation from 0 to 90°. Particularly when manufacturing elongated components, e.g. in the form of a screw or a strip-shaped mounting part, this results in particular possibilities of adaptation to the necessary strength ranges. The modules of elasticity of screws manufactured from blanks with fibers aligned axis-parallel is correspondingly higher, in other words such screws tend to be stiffer. It has been shown that the use of an extrusion process makes a change in the fiber progression as compared to the fiber progression in the blank possible, so that additional adaptation parameters become possible by means of the special fiber orientation in the blank.

In accordance with the process according to the invention, fibers which have a length of more than 3 mm can be used. In all known fiber-reinforced thermoplastic materials for manufacturing corresponding components, short fibers or long fibers are used, as a rule. The use of endless fibers with a high fiber proportion of more than 50 volume-%, in connection with the hot-forming process, results in an optimum possibility for controlling the strength properties accordingly at every point of the component to be manufactured, so that different levels of rigidity, adjusted in locally targeted manner, can be achieved.

Another characteristic of the process is that the fibers are surrounded by matrix material, covering the surface, during extrusion. This means that no additional finishing is required for the components manufactured in final form by means of the hot-forming process, since the entire surface is already practically sealed.

Within the scope of the invention, it is also possible that the components receive an additional surface seal during the hot-forming process. By means of the influence of heat in the molding die, or by additional means, e.g. coatings or release agents, an additional surface seal of the finished components can be achieved.

The hot-forming process results in various possibilities for controlling the manufacturing process. A component to be manufactured in accordance with the process according to the invention is therefore characterized by a progression of the fibers pre-determined in adaptation to the structure and the use of the component, to achieve regions with locally pre-determined rigidity and strength. The greatest tensile strength was achieved, for example, with components that were manufactured at high forming speed and high blank temperatures. Taking into consideration the torsion strength, on the other hand, maximum values are achieved if comparatively low forming temperatures and a low forming speed are used. In other words, specifically in a process for manufacturing components made of fiber-reinforced thermoplastic materials, the process according to the invention creates possibilities for adapting a component to a specific purpose of use, and it would certainly be possible to have a work step consist of two or more stages, for example, each with a different forming speed.

By means of adaptation to the shape and the use of the component, a pre-determined progression of the fibers, with reference to the longitudinal direction, diameter, thickness, shape of the component, or, in the case of openings, depressions, indentations or similar shapes in the component, regions with different fiber orientation or different fiber progression can be provided. Such a component can be particularly adapted to a special purpose of use. In other words, the force introduction and force distribution in such a component can be better adapted to the composition of the body which interacts with this component. This is particularly true for medical technology, for example in the case of bone screws and medical assembly parts and attachment strips, etc., but also for other applications in machine construction, in electrical applications or electronics, or in construction.

Therefore it is also an advantageous embodiment that this component is structured as a connection element with an engagement end for a tool and a threaded shaft, and that the rigidity of the connection element varies from the engagement end to the free end, by means of different fiber orientation. Specifically for components which can be used for the bone sector, an adaptation to the natural structure of a bone is possible, so that a light, non-magnetic, X-ray-transparent, and biocompatible connection element can be created. In contrast to most conventional metal screws, a truly effective component can be created by adaptation of the fiber structure and the fiber progression.

Furthermore, it is proposed, according to the invention, that the fibers run at least approximately parallel to the center axis of the component, from the engagement end over the thread turns which immediately follow it, while the fibers in the remaining threaded section follow the thread contour close to the surface, in the axis direction of the component, while an increasingly random distribution of the fiber orientation is provided in the core region of this section, however. This means that the greatest strength is present specifically in the region of the engagement of the component, structured as a screw, and in the subsequent threaded section, while the threaded sections which reach into the bone region have a lesser tensile strength, since no tensile forces could be absorbed specifically in this region.

In such a component according to the invention, it is also advantageous that the rigidity of the component decreases, in steps or continuously, by means of different fiber orientation from the engagement end towards the free end. Therefore a precise adaptation to the area of use of the component can be achieved specifically by means of the fiber progression, which results from the manufacturing process according to the invention and, of course, also from the forming speed.

Furthermore, it is proposed that at least one dead-end hole or one through opening, for example for inserting a turning tool or for passing through means of attachment, is provided in the component. By means of such an arrangement, it is possible to apply corresponding torsion forces when driving in such a screw-shaped component, particularly when driving it out, if this becomes necessary. In the case of through openings or similar structures, an advantageous embodiment results also for flat components, since the region surrounding the opening, for example, can be reinforced with a special fiber orientation. In this connection, it is advantageous if the dead-end hole or the through opening is molded in during manufacturing of the component. Specifically in the case of a hot-forming process, this results in special possibilities for providing corresponding dead-end holes or through openings for turning tools, specifically during a forming process.

A particular area of use for the components according to the invention results if the component is structured as a corticalis screw or spongiosa screw which is compatible in structure, for medical use.

Another exemplary embodiment of a component provides that it is formed as a strip-shaped or plate-shaped mounting part with one or more through openings and/or segments projecting beyond the length or side delimitations, where the rigidity and strength can be pre-determined over its entire length and/or width and/or diameter. In other words, it is possible to manufacture any type of component with a special structure, using the process according to the invention, where an adaptation to the necessary strength and rigidity is possible even for very specific segments, since the fiber orientation and fiber density can be pre-determined.

In this connection, it is provided that the component, structured as a mounting part, has the same strength and rigidity in the region of through openings and/or projecting segments as in other regions of the component, by means of a denser arrangement of fibers in these regions, which are usually weakened. Each component can therefore be designed in such a way that it no longer has any weakened zones, so that the strength and rigidity necessary for very specific purposes of use can be achieved in all segments.

For the strength and rigidity to be adapted in such a way, it is therefore specifically optimal if the component is structured as an osteosynthesis plate, for example for use with a corticalis screw or a spongiosa screw.

Further characteristics according to the invention and special advantages will be explained in greater detail in the specification below, on the basis of the exemplary embodiment shown in the drawing. This shows:

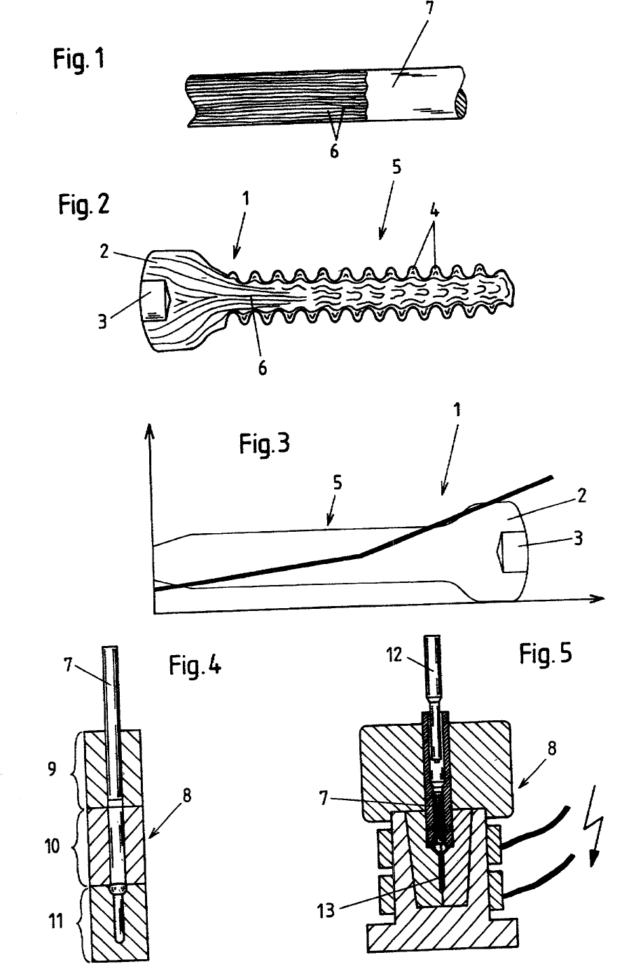

FIG. 1 a segment of a rod-shaped blank, partially shown in a cut-away view, in order to show a 0° orientation of enclosed endless fibers;

FIG. 2 a component in the form of a screw, where a schematic representation of the fiber orientation distribution in the screw is drawn in;

FIG. 3 a diagram of the progression of the rigidity, with reference to the length of the component, which is provided to be a connection element;

FIG. 4 a principle diagram of a possible melt extrusion die with temperature zones for manufacturing the component;

FIG. 5 a schematic representation of an extrusion die;

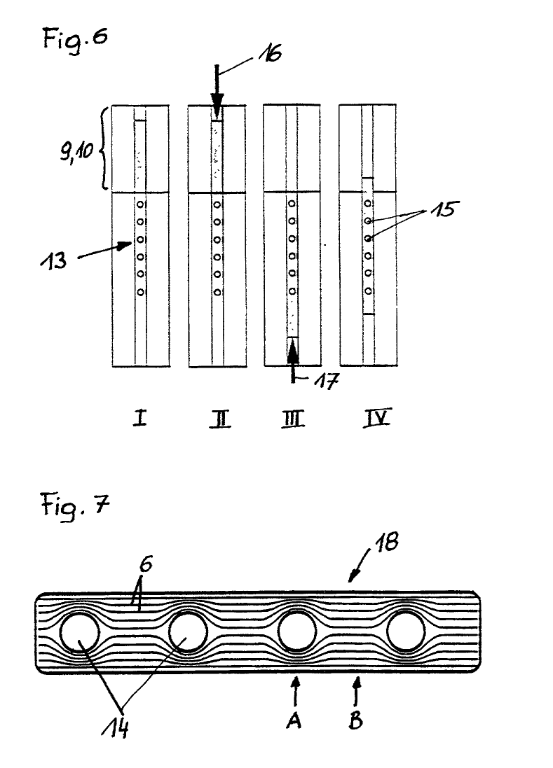

FIG. 6 a principle diagram for manufacturing a component using the push-pull extrusion process;

FIG. 7 a top view of a component manufactured using the push-pull extrusion process, which can be specifically used as an osteosynthesis plate. In the following explanation of the process according to the invention, as well as of the components manufactured according to the process, it is assumed that the component (in accordance with FIGS. 1 to 5) is a connection element, particularly a screw, which is specifically used in medical technology, in other words as a corticalis screw or spongiosa screw, for example, or that the component (in accordance with FIGS. 6 and 7) is a mounting part, particularly an osteosynthesis plate for interacting together with a connection element as mentioned above. Within the scope of the invention, of course, other components are also included, if they consist of fiber-reinforced thermoplastic materials and are manufactured in a process according to the invention. The use of such components is not limited only to medical technology. It is certainly possible to use such components also in other areas of application, such as in machine construction, in electrical technology, in aerospace technology, in civil engineering, etc. The components do not always necessarily have to be manufactured in the form of connection elements (screws), but can also be used as components with completely different design forms, such as rails or plates, for example. It would be possible, for example, to equip the components made of fiber-reinforced thermoplastic materials, which are probably not structured as self-tapping screws, with a corresponding drill section, which can also be made from biocompatible material, if necessary, or can easily be removed after the drilling process. Under some circumstances, such removal would not even be necessary in various areas of application. The example is also explained on the basis of a fiber-reinforced thermoplastic material which is produced with endless fibers with a volume proportion of more than 50%. Using the process according to the invention, however, it is just as advantageous to process fiber-reinforced thermoplastic materials which contain only short fibers or long fibers or combinations of proportions of short, long, and/or endless fibers. The process according to the invention can also be successfully used with a fiber proportion of less than 50 volume-% in the blank.

The connection element shown in the drawing, in the form of a screw 1, essentially consists of a head 2, an engagement part 3 for force introduction by a turning tool, and a shaft 5 provided with a thread 4. As is particularly evident from FIG. 2 of the drawing, the main point of the screw 2 [sic] is the progression of the endless fibers 6. By means of fibers aligned in locally targeted manner within the structure, the screw 2 [sic] has different degrees of rigidity, adjusted in locally targeted manner. This makes it possible to adapt the rigidity to the natural structure of a bone, particularly when the screw is used as a corticalis screw. By selection of a laminate of thermoplastic materials with carbon fibers, a light, X-ray-transparent, and biocompatible connection element can be created. The particular advantage of such a screw lies in the fact that the rigidity and the rigidity gradients can be better adapted to the natural structure of the bone than in the case of conventional metal screws. By means of the fiber structure, a better force distribution is guaranteed, i.e. not only the first three screw turns are the bearing parts. Furthermore, the connection element does not hinder conventional medical examination methods, since it is non-magnetic and X-ray-transparent. This is a particular disadvantage of conventional metal implants, including connection elements. They can make the examination findings of modern diagnostic methods, such as computed tomography and magnetic resonance imaging, totally useless.

Because of the post-setting behavior of the connection element, loosening would not be expected for quite some time. If the connection element is structured as a corticalis screw, the screw can be driven out again with the remaining residual strength, in case the thread has been stripped.

As already explained, the connection element can be used in corrosive environments, in general machine construction, and particularly in those cases where high strength and targeted strength with low weight are demanded. Here again, the force introduction over more than three thread turns is a deciding factor.

With the head of the corticalis screw shown in FIG. 2, various other elements can be fixed in place, for example an osteosynthesis plate. The engagement part 3 can be structured as an inside hexagonal part, for example. However, it is certainly possible to select different engagement shapes, for example a square opening, an inside star opening, or a Phillips head.

A variant of the extrusion process as known from metal processing is used to manufacture the corticalis screw (e.g. with a core diameter of 3 mm) from PAEK (polyaryl ethyl ketone) reinforced with carbon fibers. A special variant provides for the use of PEEK (polyether ethyl ketone) reinforced with carbon fibers. The fiber orientation distribution and the mechanical properties of the screw are characterized and brought into relation with the process parameters of the manufacturing process.

The fracture load of the screws manufactured using the extrusion process lies in the range between 3000 and 4000 N, the maximum torsion moment is between 1 and 1.5 Nm, where the maximum angle of distortion according to ISO standard 6475 is up 370°. The screws possess a modulus of elasticity which descreases from the head towards the tip, and can be designated as being homoelastic with the bone.

Nature frequently utilizes the principle of fiber reinforcement in its structures. It is therefore advantageous, for reasons of structural compatibility, to structure medical implants also as fiber laminate parts. Particularly in the field of osteosynthesis technology, developments are necessary to replace conventional steel osteosynthesis plates with less rigid implants made of fiber laminate materials. Specifically in connection with osteosynthesis plates, the structure according to the invention has an advantageous effect. Such an osteosynthesis system has numerous advantages as compared with a conventional steel implant. For one thing, there is homoelasticity relative to the bone, and therefore adapted load introduction into the bone is possible; for another thing, X-ray-transparency and computed tomography are possible. Furthermore, the measures according to the invention also result in cost-effective production in a hot-forming process. In addition, the fact that components structured in such a way are unproblematic in cases of nickel allergies is an additional point.

In research work in this field, it was found that only when using bone screws made of thermoplastic materials reinforced with carbon fibers and, in this connection, by means of the manufacturing process according to the invention, was it possible to create an optimum variant. Based on the extrusion process developed in this connection, bone screws made of PEAK [sic] reinforced with carbon fibers were manufactured and characterized.

In the extrusion of metal parts, the work piece is generally pressed into a die at room temperature, using a punch. This is therefore one of the so-called press-through processes according to DIN 8583. For processing the fiber-reinforced thermoplastic materials, the process was modified in that the blank element is not formed at room temperature, but rather above the melting temperature of the matrix material.

Round rods 7 of PAEK reinforced with carbon fibers (FIG. 1) serve as blanks for screw manufacturing; they have a fiber volume content of more than 50%, preferably 60%, where two different blank types were used with regard to the fiber orientation, namely blanks with a purely axis-parallel fiber orientation, on the one hand, and blanks with a fiber orientation between 0 and ±90°, on the other hand.

A blank element is heated to the forming temperature (e.g. 350-450° C.) in a heated extrusion die 8 (heating stage), where heating can also take place in consecutive heating stages 9 and 10 (FIG. 4). The blank 7 is therefore brought into the first heating stage 9, pre-heated accordingly there, heated further in the heating stage 10, and then formed in the negative mold in the region of stage 11. By means of the punch 12, the blank 7 is pressed into the negative mold (mold cavity) 13, and receives its final shape there. The pressing speed can be in the range between 2 and 80 mm/s in this connection. The pressing pressure was 120 MPa in various tests. During a subsequent post-pressure stage pressure approximately 90 MPa), the die is cooled below the glass transition temperature of PAEK (143° C.), using compressed air. After the extrusion die is opened, the finished corticalis screw can be removed. In a subsequent analysis of a screw manufactured in this manner, it was shown that optimum values can be achieved in each instance. This results from the high proportion of fibers, the use of endless fibers, and the very specific forming process for manufacturing the screw. As is evident from FIG. 2, the fibers are aligned predominantly in the direction of the screw axis in the region of the head 2 of the screw 1. In the region of the screw tip, the fibers follow the screw contour (in other words the thread progression) in the edge region, while a random distribution of the fibers orientation prevails in the core zone.

With regard to the mechanical properties, it must be stated that the mean value of tensile strength of the corticalis screws is about 460 N/mm2. The greatest tensile strength was achieved with screws which were manufactured at high forming speeds (approximately 80 mm/s) and high blank temperatures (approximately 400° C.). The torsion strength of screws which were manufactured from blanks with an axis-parallel fiber orientation is 18% higher, on average, than for screws made from blanks with a 0°−/±45° fiber orientation. The maximum values were measured for screws which were manufactured at relatively low temperatures (380° C.) and low forming speeds (2 mm/s). The modulus of elasticity in the lengthwise direction of the screw is not constant, but rather decreases greatly towards the tip. The moduli of elasticity vary between 5 and 23 GPa, where screws which were manufactured from blanks with a 0° fiber orientation tend to be stiffer. This is also clearly evident from the schematic diagram according to FIG. 3. The rigidity represented by the diagram line increases in the direction of the screw head, where a bend exists in this line, specifically in a certain region of the length of the shaft 5 with a thread. Specifically in this region, as is also evident from FIG. 2, the axis-parallel fiber orientation provided in the core region comes to an end.

Using the example of a corticalis screw, it has been shown that components with complex geometry can also be manufactured by extrusion of thermoplastic materials reinforced with long fibers, in a hot-forming process. The fiber orientation distribution as the defining variable for the mechanical properties can be controlled, within certain limits, by means of a suitable selection of the fiber orientation in the blank. The other process parameters investigated (forming speed and forming temperature) have a lesser influence on the extrusion result.

The tensile strength of extrused PEAK lies about 30% below that of comparable steel screws, on average. An average fracture strength of 3200 N is sufficient for osteosynthesis applications, since a corresponding screw is already pulled out of the bone at a tensile force of 800-1300 N.

The ISO standard 6475 requires a minimum fracture moment of 4.4 Nm and a torsion angle of at least 180° for steel screws with comparable dimensions. Such requirements cannot be met with screws made of fiber-reinforced thermoplastic materials (maximum 1.3 Nm). However, experiments have shown that stripping of threads and therefore destruction of the screw while it is being driven into the bone is precluded, since the thread was already destroyed in the bone at a torque of approximately 0.8 Nm. The slow decrease in residual strength after primary failure would permit the damaged screw to be driven out of the bone even after a fracture.

With a modulus of elasticity between 5 and 23 GPa, the extruded corticalis screw is similar to the bone in its elastic behavior. The rigidity in the lengthwise direction clearly decreases towards the tip (decreasing rig radient). In the screwed-in state, the rigid part of the screw (head region) is therefore close to the corticalis and therefore at the most rigid part of the treated bone. With such a rigidity distribution, a force introduction which is extensively adapted to the bone structure can be achieved.

With the present invention, the possibility has been created, for the first time, of manufacturing components from fiber-reinforced thermoplastic materials, which components have a special structure of a thread, a head, a shape, etc., for example, and are manufactured using a hot-forming process, and of achieving a design compatible with the area of application via the material properties, particularly the precise alignment of fibers.

In the process description, the point of departure was an extrusion process which is practically effective only in one direction. In this process, the blank is brought to a corresponding temperature (dough-like or honey-like flowing consistency) and then pressed into a negative mold. Within the scope of the invention it is also possible to use a push-pull extrusion process, specifically for manufacturing strip-shaped, rail-shaped, or plate-shaped parts, but also for screw-like or other connection elements and also for special shapes of parts or for special structures of bolts, etc. Under some circumstances, a desired fiber orientation and fiber distribution can be achieved by multiple pressing back and forth, in other words by a multiple reversal of the pressing direction. Additional details in this regard will be explained at greater length on the basis of FIG. 6 and 7. The push-pull extrusion process can be of specific importance if, for example, dead-end holes, through openings, indentations, or special shapes are provided in the corresponding part. Then the special progression of the fibers can be influenced, and the component to be manufactured can therefore be particularly reinforced specifically in that region where special reinforcement is necessary.

As a coating in the use of the process according to the invention, the use of carbon or graphite is provided. These coatings or release agents have been utilized practically only for the metal sector until now, and not for plastics. This results in additional advantages, since graphite is biocompatible, in contrast to the usual release agents for plastic.

In FIG. 2, an opening which is only short when viewed in an axial direction is provided for an engagement part 3. Within the scope of the invention, it is also possible to provide a correspondingly deeper dead-end hole or an opening which goes through axially here, in order to insert a corresponding turning tool. This would make it possible to overcome a higher driving-in torque, in addition to the values of torsion strength which already exist, since a corresponding tool can be inserted into correspondingly long insertion channels. Since manufacturing of such a screw takes place using the extrusion process according to the invention, this additional shaping is possible without problems.

Specifically in the case of rails or plates, through openings, indentations, dead-end holes, etc., can be provided, and these are then specifically surrounded by fibers.

The fiber orientation in the screw 1 according to FIG. 2, or in a corresponding different component for another area of use, must fundamentally be considered in differentiated manner. It is specifically possible, using the measures according to the invention and the process according to the invention, to allow optimum fiber orientation in the finished component for each special purpose of use. Particularly in the case of a high fiber proportion of more than 50 volume-% and when using endless fibers, particularly effective variants are obtained in many areas of technology, particularly in the sector of connection elements and in the sector of medical technology.

FIG. 6 shows a push-pull extrusion process, in a schematic representation, where the consecutive process steps I to IV are evident. In Step I, the blank 7 is inserted into a heating stage (section 9, 10) and heated to the forming temperature there. In Step II, the blank is pressed into the negative mold 13 in the direction of the arrow 16. In Step III, the blank 7, which has already been formed once, is pressed back again in the opposite direction (direction of the arrow 17). In Step IV, the blank, which has been formed twice or multiple times, is end-compressed, cooled and unmolded, to produce the finished component.

By means of bolts 15 which is inserted into the negative mold 23 or pass through it, components with through openings 14 can be manufactured, where the black is pressed past these bolts 15 several times during the course of the push-pull extrusion process. This results in a very special progression of the fibers 6, as is evident from FIG. 7. The same or a similar effect is also obtained if projecting sections were present at the length and/or side delimitations of the component, structured as a mounting part 18. In the zones A, which are usually weakened, this results in a denser arrangement of the fibers 6, so that the same strength or rigidity is obtained in these zones as in the other regions B of such a component.

Such an embodiment of a component is excellently suited for osteosynthesis plates, which can then be used, for example, in interaction with a screw manufactured by the process according to the invention. Of course, the same advantages of biocompatibility apply to these plates, and in addition, the strength and rigidity are by no means less than that of the plates mainly used until now, which are made of stainless steel.

In push-pull extrusion, various additional parameters are possible, by means of which the predictability of the fiber progression and therefore the adaptation of strength and rigidity to the structure of the component can be further improved. For example, the number of push or pull cycles, the cycle length, the cycle speed, the pressure and counter-pressure can be adjusted. Steps II and III can be repeated as often as desired, and for each push or pull cycle, the cycle path length can be newly selected. Centering of the component during Step IV does not necessary have to take place. All the parameters can be changed as desired in Steps II to IV.

The endless fibers are not excessively stressed during such a process, so that they do not break in many places. The transition from sites with strongly aligned fibers and sites with a homogeneous fiber distribution is continuous. In contrast to a known lamination technique, the process makes it possible to produce components which are not in sheet form. Geometries which otherwise occur only in injection-molding are made possible. In this connection, significantly higher strengths are actually achieved according to the invention. It has also become possible to manufacture components with holes, undercuts, etc. It is possible to optimize the fiber orientation in such a way that the capacity of the fibers, for example with regard to the mechanical properties, is fully utilized. The process allows composite processing which is in keeping with endless fiber reinforcement. In a single component, sites with isotropic and anisotropic properties occur next to one another, without any border surface being present. Since border surfaces or seams are also weak points, the invention also reduces the susceptibility of the component to fatigue.

In the push-pull extrusion process according to the present invention, other variants are also possible. For example, a cycle step could be carried out not only in one direction, but also using two or three main axes. Furthermore, it would be possible to insert the pins shown in FIG. 6 only after homogenization of the blank, in other words after one or more Steps II or III. It would also be possible to have a previously homogenized blank, which has already been formed once or several times in a previous station.

Within the scope of the invention, it is also possible to use blanks which consist of layers with different fiber orientation that run in the lengthwise direction of the blanks. It would also be possible to use a blank consisting of more than one polymer laminate (also when first producing rod material with any desired cross-section). In such a case, the blank could consist of several layers with different matrix material and/or different arrangements and/or different volume-% proportions and/or different fiber materials and/or different lengths of the fibers. If endless fibers are used, then these generally have a length which at least corresponds to the length of the blank 7, as it is cut off from the rod material, in adaptation to the finished component.

1. Process for manufacturing components made of fiber-reinforced thermoplastic materials, where a blank (7) formed of a short, long, and/or endless fiber (6) and a thermoplastic material is first pre-finished, and this blank (7) is brought into the final form of the component in a negative mold, under pressure, in a hot-forming process, characterized in that the blank (7) is first heated to forming temperature in a heating stage, and then pressed into the negative mold (13) by means of extrusion.

2. Process for manufacturing components which are under tensile, bending, and/or torsion stress, made of fiber-reinforced thermoplastic materials, where a blank (7) formed with a fiber proportion of more than 50 volume-% and with at least predominant use of endless fibers and a thermoplastic material is first pre-finished, and this blank is brought into the final form of the component in a negative mold, under pressure, in a hot-forming process, characterized in that the blank (7) is first heated to forming temperature in a heating stage, and then pressed into the negative mold (13) by means of extrusion.

3. Process according to claim 1 or 2, characterized in that the blank (7) is pre-finished as rod material and is cut to the lengths required for the final component before the hot-forming process.

4. Process according to claim 1 or 2, characterized in that endless fibers (6) with a length that corresponds at least to the length of the blank for the final component are used.

5. Process according to one of claims 1 to 4, characterized in that a blank (7) composed of layers with different fiber orientation in its lengthwise direction is formed.

6. Process according to one of claims 1 to 4, characterized in that a blank (7) is formed from more than one polymer laminate, e.g. with several layers with a different matrix material and a different arrangement and/or different volume-% proportion and/or different fiber material and/or different lengths of the fibers.

7. Process according to one of claims 1 to 6, characterized in that the blank (7) is formed into the final component by means of a push-pull extrusion process.

8. Process according to one of claims 1 or 7, characterized in that the blank (7) is heated to a forming temperature of 350-450° C., for example, in a heating stage and then pressed into the negative mold (13), where cooling below the glass transition temperature of the thermoplastic material, e.g. 143° C., takes place during a post-pressure phase.

9. Process according to one of the preceding Claims, characterized in that during the hot-forming process, carbon or graphite is used as a release agent.

10. Process according to one of the preceding Claims, characterized in that a blank (7) made of PAEK (polyaryl ether ketones) reinforced with carbon fibers (6) is processed.

11. Process according to one of claims 1 to 10, characterized in that at least part of the endless fibers (6) run parallel to the axis of the blank (7).

12. Process according to one of claims 1 to 11, characterized in that at least a portion of the fibers (6) have an orientation from 0 to 90° in the blank (7).

13. Process according to one of claims 1 to 12, characterized in that the fibers (6) have a length of more than 3 mm.

14. Process according to one of claims 1 to 13, characterized in that the fibers are surrounded by matrix material, covering the surface, during extrusion.

15. Process according to one of claims 1 to 14, characterized in that the pressing temperature and the pressing speed are adjusted as a variable to change the position and the alignment of the fibers in the finished component.

16. Process according to one of claims 1 to 15, characterized in that the components receive an additional surface seal during the hot-forming process.

17. Component made of fiber-reinforced thermoplastic materials, manufactured by a processes according to at least one of claims 1 to 16, characterized by a progression of the fibers pre-determined in adaptation to the structure and the use of the component, to achieve regions with locally pre-determined rigidity and strength.

18. Component according to claim 16, characterized in that this component is structured as a connection element with an engagement end for a tool and a threaded shaft (5), and that the rigidity of the connection element varies from the engagement end to the free end, by means of different fiber orientation.

19. Component according to claim 17 or 18, characterized in that the fibers (6) run at least approximately parallel to the center axis of the component, from the engagement end over the thread turns (4) which immediately follow it, while the fibers (6) in the remaining threaded section follow the thread contour close to the surface, in the axis direction of the component, while an increasingly random distribution of the fiber orientation is provided in the core region of this section.

20. Component according to claims 18 and 19, characterized in that the rigidity of the component decreases, in steps or continuously, by means of different fiber orientation from the engagement end towards the free end.

21. Component according to one of claims 16 to 20, characterized in that at least one dead-end hole or one through opening, for example for inserting a turning tool or for passing through means of attachment, is provided in the component.

22. Component according to claim 21, characterized in that the dead-end hole or the through opening is molded in during manufacturing of the component.

23. Component according to one of claims 17 to 22, characterized in that the component is structured as a corticalis screw or spongiosa screw which is compatible in structure, for medical use.

24. Component according to claim 17, characterized in that it is formed as a strip-shaped or plate-shaped mounting part (18) with one or more through openings (14) and/or segments projecting beyond the length or side delimitations, where the rigidity and strength is pre-determined over its entire length and/or width and/or diameter.

25. Component according to claims 17 to 24, characterized in that the component, structured as a mounting part (18), has the same strength and rigidity in the region of through openings (14) and/or projecting segments as in other regions of the component, by means of a denser arrangement of fibers (6) in these regions, which are usually weakened.

26. Component according to claims 17, 24 or 25, characterized in that the component is structured as an osteosynthesis plate, for example for use with a corticalis screw or a spongiosa screw.
